(19) United States
(12) Patent Application Publication
(10) Pub. No.: US 2024/0345119 A1
(43) Pub. Date: Oct. 17, 2024

(54) AUTOMATIC ANALYSIS SYSTEM

(71) Applicant: Siemens Healthcare Diagnostics Products GmbH, Marburg (DE)

(72) Inventor: Juergen Patzke, Marburg (DE)

(21) Appl. No.: 18/619,034

(22) Filed: Mar. 27, 2024

(30) Foreign Application Priority Data
Mar. 27, 2023 (EP) 23164272

(51) Int. Cl. G01N 35/04; G01N 35/00

(52) U.S. Cl. CPC G01N 35/04; G01N 2035/00346; G01N 2035/0434

(57) ABSTRACT

An automatic analysis system (1) for analyzing a sample (23), the analysis system (1) comprising a first device (2) for storing cartridges (3) at a first temperature (T1), the first temperature (T1) being less than 268.15 kelvin.

### CROSS REFERENCE TO RELATED APPLICATION

This claims priority to European Patent Application No. EP 23164272.9, filed Mar. 27, 2023, which is hereby incorporated by reference herein in its entirety for all purposes.

### FIELD

The invention relates to an automatic analysis system for analyzing a sample, having a device for storing cartridges.

### BACKGROUND

Many detection and analysis methods for the determination of physiological parameters in body fluid samples or other biological samples are nowadays carried out in an automated fashion in a large number in automatic analysis instruments, also so-called in vitro diagnostic systems.

Modern analysis instruments are capable of carrying out a multiplicity of detection reactions and analyses with one sample. In order to be able to carry out a multiplicity of investigations in an automated fashion, various devices for the physical transfer of measurement cells, reaction containers and reagent containers are required, for example, transfer arms with a grip function, conveyor belts or rotatable transport wheels, as well as devices for the transfer of liquids, for example, pipetting devices. The instruments comprise a control unit which, by means of corresponding software, is capable of planning and performing the working steps for the desired analyses substantially independently.

Many of the analysis methods used in such automated analysis instruments are based on optical methods. These methods allow the qualitative and quantitative detection of analytes, i.e., the substances to be detected or determined in samples. The determination of clinically relevant parameters, for example, the concentration or activity of an analyte, frequently takes place by mixing a part of a sample with one or more test reagents in a reaction vessel, which may also be the measurement cell, thereby initiating, for example, a biochemical reaction or a specific binding reaction which causes a measurable change of an optical or other physical property of the test preparation.

In medical technology instruments, for example, diagnostic analysis instruments for the automatic analysis and investigation of, for example, in vitro samples of biological body fluids, a measurement cuvette is, for example, filled with the required reagents by means of a pipetting device with a pipetting needle. The measurement cuvette is automatically moved with a cuvette gripper to different positions inside the automatic analysis instrument by means of a robot arm, which is part of a robot station. After the measurement, the used measurement cuvette is brought into a refuse container for disposal through a refuse chute.

In such automatic analyzers, consumable materials, for example, reagents, controls and standards, are stored in the instrument in order to allow automatic operation over a relatively long time or the analysis of a relatively large number of samples.

Developing special tests, which are typically requested only rarely in a laboratory, for existing, fully automatic analysis systems is generally comparatively expensive and therefore often economically unviable.

Already established tests available on the market can generally be adapted to the new analysis systems only with a large number of elaborate technical modifications. This therefore usually entails high development costs. The currently conventional, fully automatic analysis systems are designed for tests which are run frequently, so that it is profitable to keep reagents, controls and standards in, for example, cartridges or flasks on the instrument.

For tests which are carried out rarely, the stability time of these components is often exceeded, and so they regularly have to be discarded, which causes additional costs and further makes automated performance of the tests uneconomical. Further, calibrations need to be carried out comparatively often for such tests, sometimes for each individual sample measurement, which is technically demanding, time-intensive and economically disadvantageous.

To date, there have essentially been on the one hand fully automatic analysis systems which are technically optimized for tests to be carried out frequently, and on the other hand either point-of-care tests or ELISAs (enzyme-linked immunosorbent assays) or other solutions with various manual steps, but no fully automated solution for very infrequent, special tests.

The devices from the prior art thus usually do not allow simple and economically viable development and provision of tests requested only rarely in a laboratory on fully automatic analysis systems.

### SUMMARY

It is therefore an object of the invention to allow simplified development and provision of tests to be carried out on fully automated analysis systems, in particular, even for tests requested only rarely in a laboratory.

This object is achieved according to the invention by the subjects and methods described below.

It has been found that improved, simplified development and provision of tests for carrying out tests requested only rarely in a laboratory on fully automated analysis systems can be achieved by use of frozen reagents which are provided in a cartridge together with measurement cuvettes.

Cartridges adapted to the test in question are used in this case, as well as frozen reagents and/or controls and measurement cuvettes, all these components being contained in the cartridge.

This has the advantage that the development outlay can be reduced significantly in particular for tests requested only rarely in a laboratory, and at the same time such tests can be provided for fully automated analysis systems and kept and carried out in laboratories in an optimized fashion.

The present invention provides in particular an automatic analysis system for analyzing a sample, the analysis system comprising a first device for storing cartridges at a first temperature, the first temperature being less than 268.15 kelvin, preferably a second device for receiving a transport box, a third device for storing cartridges at a second temperature, the second temperature being more than 273.15 kelvin, a fourth device for heating the cartridges from the first temperature to the second temperature, a fifth device for pipetting at least one liquid, a sixth device for incubating at least one cartridge, a seventh device for measuring at least one sample preparation of the sample, wherein the cartridges each comprise at least one first compartment with at least one reagent, at least one second compartment with at least one control, and at least one third compartment, the third compartment comprising a measurement cuvette, wherein a measurement of the test preparation can take place by means of the seventh device in the measurement cuvette, wherein at least one cartridge can be delivered to the analysis system, wherein the cartridge has a third temperature, the third temperature being less than 268.15 kelvin, wherein the transport box is preferably configured to transport at least one cartridge at the third temperature.

Preferably, the cartridge is delivered to the analysis system in the transport box. The cartridge is thus preferably contained at least partially, preferably fully, inside the transport box during the delivery into the analysis system. Preferably, the transport box is correspondingly delivered to the analysis system with one or more cartridges therein.

The third device is preferably configured as a block, which is preferably regulated as a whole to 37 degrees Celsius. This block preferably contains a separate apparatus, for example, a water bath and/or a block of metal, which is used to thaw the cartridges and preferably comprises or constitutes the fourth device.

The third device preferably further comprises an incubation block for the sample preparation, in which case the incubation may, for example, take place in air, in a water bath and/or a block of metal which comprises or constitutes the sixth device.

The third device preferably further comprises a measuring unit in which the measurement of the sample preferably takes place in an air space, that is to say the sample is preferably not contained in a water bath and/or a block of metal during the measurement. The seventh device preferably comprises or constitutes this measuring unit.

Preferably, the first temperature and the third temperature are equal, although they may also be different. The latter is particularly advantageous if the return transport and/or further transport of the cartridges takes place at room temperature.

In one preferred embodiment, the transport box is further configured to transport at least one cartridge at a fourth temperature, the fourth temperature being 233.15 to 323.15 kelvin, preferably 288.15 to 298.15 kelvin, particularly preferably room temperature.

In another preferred embodiment, the third device at least partially, preferably fully, comprises the fourth device, the fifth device, the sixth device and/or the seventh device. The third device thus advantageously comprises various other devices and integrates them into one device.

In another preferred embodiment, the second temperature is a predetermined, constant temperature between 293.15 and 315.15 kelvin, preferably between 308.15 and 312.15 kelvin, the second temperature particularly preferably being 310.15 kelvin.

In another preferred embodiment, the first temperature is less than 268.15 kelvin, preferably less than 263.15 kelvin, particularly preferably 255.15 to 245.15 kelvin.

In another preferred embodiment, the liquid comprises a sample liquid.

In another preferred embodiment, the fifth device comprises a pipetting needle for pipetting samples, reagents, wash solutions and/or a dilution medium, the dilution medium preferably comprising a buffer solution.

In another preferred embodiment, the second device comprises a recess, the recess being configured to receive at least one transport box.

In another preferred embodiment, the analysis system comprises an eighth device for controlling the first, second, third and/or fourth temperature.

In another preferred embodiment, the analysis system comprises a ninth device for automatically bringing at least one cartridge out of a transport box contained in the second device and into the first device.

In another preferred embodiment, the analysis system comprises a tenth device for automatically bringing at least one cartridge out of the first device and into the third device.

In another preferred embodiment, the analysis system comprises an eleventh device for automatically bringing at least one cartridge out of the third device into a transport box contained in the second device.

Another preferred subject of the invention is, in particular, a transport box for an automatic analysis system according to the invention for transporting at least one cartridge to the analysis system and/or from the analysis system, wherein the cartridge comprises at least one first compartment with at least one frozen reagent, at least one second compartment with at least one control, and at least one third compartment, the third compartment comprising a measurement cuvette, the transport box preferably containing at least one cartridge.

Another subject of the invention is, in particular, a cartridge which can be transported, preferably by means of a transport box according to the invention, to an automatic analysis system according to the invention and/or from the analysis system, wherein the cartridge comprises at least one first compartment with at least one frozen reagent, at least one second compartment with at least one control, and at least one third compartment, the third compartment comprising a measurement cuvette.

Another subject of the invention is, in particular, the use of a transport box according to the invention and/or a cartridge according to the invention in an automatic analysis instrument according to the invention.

Another subject of the invention is, in particular, a method for analyzing a sample by means of an automatic analysis system according to the invention by using a cartridge according to the invention and preferably a transport box according to the invention, the method comprising the following steps:
delivering the cartridge, preferably in the transport box, to the automatic analysis system, the cartridge having the first temperature,
preferably bringing the transport box with the cartridge into the second device,
preferably removing the cartridge from the transport box and delivering it into the first device by means of the ninth device, and/or delivering the cartridge (5) into the first device (2),
removing the cartridge from the first device and delivering it into the third device by means of the tenth device,
heating the cartridge from the first temperature to the second temperature by means of the fourth device,
pipetting at least one sample, at least one reagent and at least one control by means of the fifth device comprising the production of a test preparation,
preferably incubating the test preparation in the cartridge by means of the sixth device,
measuring the test preparation by means of the seventh device in the measurement cuvette of the third compartment of the cartridge,
evaluating the measurement of the test preparation by using at least one calibration curve, which has preferably been transmitted electronically to the analysis system,
preferably bringing the cartridge out of the third device into the transport box by means of the eleventh device,
preferably transporting the cartridge in the transport box to a disposal station, the cartridge having the fourth temperature.

Preferably, the delivery of the cartridge (5) into the first device (2) takes place manually and/or directly and preferably on a direct path, without the cartridge (5) previously being delivered into another device of the analysis system.

In one preferred embodiment, the method comprises carrying out a hemostasis test, preferably a factor VIII binding test.

In other preferred embodiments, the method comprises carrying out one or more of the tests and/or groups of tests which are described in chapter 16 “Hämostasesystem” [“Hemostasis system”] under sections 16.10 to 16.28 in “Labor und Diagnose: Indikation und Bewertung von Laborbefunden für die medizinische Diagnostik” [“Laboratory and diagnosis: indication and evaluation of laboratory data for medical diagnostics”] by Lothar Thomas, 8th edition 2012, ISBN-10:3980521583, ISBN-13:978-3980521581, and/or in the chapters “Laboratory investigations” in the publication “Clinical Laboratory Diagnostics” available electronically online at https://www.clinical-laboratory-diagnostics.com by Lothar Thomas, released 12.15.2022.

In other preferred embodiments, the method comprises carrying out one or more of the tests and/or groups of tests specified below.

ELISA test for determining the concentration of the thrombin-antithrombin complex as a marker for prothrombotic states (Pelzer, Hermann, Angela Schwarz, and Norbert Heimburger. “Determination of human thrombin-antithrombin III complex in plasma with an enzyme-linked immunosorbent assay.” Thrombosis and Haemostasis 59.01 (1988): 101-106.).

ELISA test for determining ADAMTS13 activity with anti-N10 monoclonal antibodies to identify a thrombotic thrombocytopenia purpura (Kato, Seiji, et al. “Novel monoclonal antibody-based enzyme immunoassay for determining plasma levels of ADAMTS13 activity.” Transfusion 46.8 (2006): 1444-1452.).

Test for determining protein S-TFPI cofactor activity by means of measuring thrombin generation (Alshaikh, N. A., et al. “New functional assays to selectively quantify the activated protein C- and tissue factor pathway inhibitor-cofactor activities of protein S in plasma.” Journal of Thrombosis and Haemostasis 15.5 (2017): 950-960.).

Ecarin chromogenic assay test to quantify the anticoagulant effect of direct thrombin inhibitors (Lange, U., G. Nowak, and E. Bucha. “Ecarin chromogenic assay—a new method for quantitative determination of direct thrombin inhibitors like hirudin.” Pathophysiology of haemostasis and thrombosis 33.4 (2003): 184-191.).

The test dFiix-PT to determine the anticoagulant effect of warfarin, dabigatran, rivaroxaban and apixaban (Letertre, L. R., et al. “A single test to assay warfarin, dabigatran, rivaroxaban, apixaban, unfractionated heparin, and enoxaparin in plasma.” Journal of Thrombosis and Haemostasis 14.5 (2016): 1043-1053.).

In preferred embodiments, no incubation of the test preparation takes place, or any desired number of incubation steps take place, for example, one to ten incubation steps. These may, in particular, also include steps of incubating reagent mixtures which do not yet contain samples, i.e., the test preparation in such an incubation step consists of a reagent and/or a reagent mixture and does not yet comprise a sample.

According to the invention, tests may require calibrations. Preferably, calibrations take place by means of one-point and/or timepoint calibrations, which means that a calibration curve shape is provided electronically and the location of the calibration curve is established by measuring one single or at most two measurement points. The cartridges preferably comprise one or more calibrators for carrying out one or more calibrations. The calibrators are preferably arranged in one or more compartments of the cartridge, which are preferably configured specifically for this purpose.

In another preferred embodiment, the method further comprises the following step:
automatically reordering used cartridges.

In another preferred embodiment, the method further comprises the following step:
automatically determining which tests and/or how many cartridges are to be kept in the analysis system, and preferably correspondingly automatically ordering and/or reordering tests and/or cartridges.

A “sample” in the context of the invention means the material that is suspected to contain the substance to be detected (the analyte). The term “sample” includes, in particular, biological fluids of humans or animals, for example, blood, plasma, serum, sputum, exudate, bronchoalveolar lavage, lymph, synovial fluid, semen, vaginal mucus, feces, urine, cerebrospinal fluid or, alternatively for example, tissue samples or cell culture samples correspondingly prepared by homogenization or cell lysis for photometric, preferably nephelometric, determination. Furthermore, for example, vegetable fluids or tissue, forensic samples, water samples and effluent samples, foodstuffs, pharmaceuticals, which may be subjected to a corresponding sample preparation before the determination, may also be used as sample.

A “reagent” in the context of the invention means a material which contains substances that, upon contact with other substances from reagents and substances from the sample, induce specific reactions which are physically measurable and can be used to measure an analyte from a sample. In particular, reagents also include buffers, aqueous NaCl solutions and/or single or multiple distilled water.

A “buffer” in the context of the invention means a material which is used only, or among other things, to keep the pH constant in the reaction preparation. In particular, buffers also include aqueous NaCl solutions and/or single or multiple distilled water.

In a quantitative detection, the amount, the concentration or the activity of the analyte in the sample is measured. The term analyte also includes parameters which register the functional paths of metabolic processes, for example, activated partial thromboplastin time (APTT). The term “quantitative detection” also includes semiquantitative methods which only record the approximate amount, concentration or activity of the analyte in the sample or can only be used for a relative quantity, concentration or activity indication. Qualitative detection means the detection of the presence of the analyte in the sample at all or the indication that the amount, concentration or activity of the analyte in the sample lies below or above a particular threshold value or a plurality of particular threshold values.

A measurement cuvette is, for example, a cuvette or a reaction vessel made of glass, plastic or metal. The measurement cuvette is advantageously made from optically transparent materials, which may be advantageous particularly when using optical analysis methods.

The terms “measurement cuvette” and “cuvette” are used synonymously and refer to the same object.

### BRIEF DESCRIPTION OF THE DRAWINGS

The invention will be explained in more detail by way of example with the aid of drawings, in which:

FIG. 1 schematically shows the structure of an automatic analysis system (1), a cartridge (3), a transport box (5) and a central storage unit (25);

FIG. 2 schematically shows details of a cartridge (3) which can be transported by means of the transport box (5);

FIG. 3 schematically shows a method (30) for analyzing a sample (23) by means of an automatic analysis system (1).

In all figures, parts which are the same are provided with the same reference signs.

### DETAILED DESCRIPTION

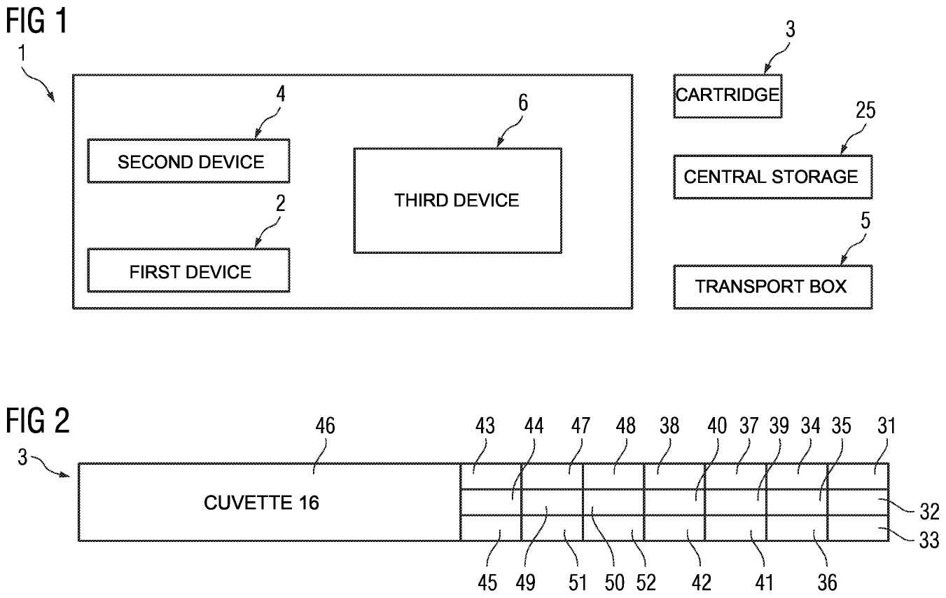

FIG. 1 shows an automatic analysis system (1), a cartridge (3), a transport box (5) and a central storage unit (25). The automatic analysis system (1) is configured to automatically analyze a sample (23). The analysis system (1) comprises a first device (2) for storing cartridges (3) at a first temperature (T1). The first temperature (T1) is less than 268.15 kelvin. The automatic analysis system (1) further comprises a second device (4) for receiving a transport box (5) and a third device (6) for storing, or using according to the invention, cartridges (3) at a second temperature (T2). The second temperature (T2) is more than 273.15 kelvin. The third device (6) comprises a fourth device (7) for heating the cartridges (3) from the first temperature (T1) to the second temperature (T2), a fifth device (8) for pipetting at least one liquid, a sixth device (9) for incubating at least one cartridge (3) and a seventh device (10) for measuring at least one sample preparation (11) of the sample (23). The fourth device (7), fifth device (8), sixth device (9) and seventh device (10), the sample (23) and the sample preparation (11) are not represented.

FIG. 1 further schematically shows a cartridge (3) according to the invention which can be transported by means of a transport box (5) according to the invention. The cartridge (3) in each case comprises a first compartment (12) with a frozen reagent (13), a second compartment (14) with a control (15), and a third compartment (16). The third compartment (16) comprises a measurement cuvette (17). A measurement of the test preparation (11) takes place according to the invention by means of the seventh device (10) in the measurement cuvette (17). The compartments (12, 14, 16), the reagent (13), the control (15) and the measurement cuvette (17) are not represented.

FIG. 1 further schematically shows a transport box (5) according to the invention. The transport box (5) is configured to transport at least one cartridge (3) at a third temperature (T3). The third temperature (T3) is less than 268.15 kelvin.

FIG. 1 further schematically shows a central storage unit (25) according to the invention.

FIG. 2 schematically shows details of a cartridge (3) according to the invention, which is configured in such a way that it can be transported by means of a transport box (5) according to the invention.

The cartridge (3) is shown in a plan view and various compartments are schematically represented. The dimensions of the cartridge (3) are 100 mm in length, 20 mm in width and 15 mm in height. The cartridge (3) comprises 22 individual cuvettes (31 to 52) 1 to 22, of which 21 cuvettes (31 to 45 and 47 to 52) are configured identically, particularly in size and shape. These 21 cuvettes (31 to 45 and 47 to 52) are arranged in the shape of a block in plan view in three rows of 7 cuvettes each on the right in the cartridge (3) and occupy half the volume of the cartridge (3). A further cuvette (46) occupies the other half of the volume of the cartridge (3) on the left in plan view. The cuvettes (31 to 52) each have an opening at the top over substantially the entire cross section of the respective cuvette.

One application example according to the invention for the automatic analysis system (1), the transport box (5) and the cartridge (3) is carrying out a factor VIII binding test, which is configured as an enzyme-linked immunosorbent assay (ELISA). The factor VIII binding test measures the capability of the von Willebrand factor to bind factor VIII and is necessary in order to diagnose subtype 2N of von Willebrand disease. This subtype occurs very rarely, but subtype differentiation for von Willebrand disease is very important in order to make the correct therapy decisions. There is to date no fully automatic test, but instead usually only tests in the ELISA format. These are comparatively expensive to purchase and perform. Performing them has to date partially involved manual steps, as a result of which high personnel costs may also be entailed. Usually, a calibration curve elaborately has to be recompiled for each run in multiple determination. The accuracy of the tests may also sometimes be in need of improvement.

Such a factor VIII binding test may, for example, be a diagnosis method involving the kit ASSERACHROM VWF:FVIIIB from Diagnostica Stago S.A.S. (France, package insert version February 2018, REF 00919, https://ifu.stago.com/fileadmin/user_upload/notices/Notices_Rea ctifs/0091904201802/DE_ASSERACHROM%23VWF%23FVIIIB_20180228.pdf downloaded on 03.16.2023).

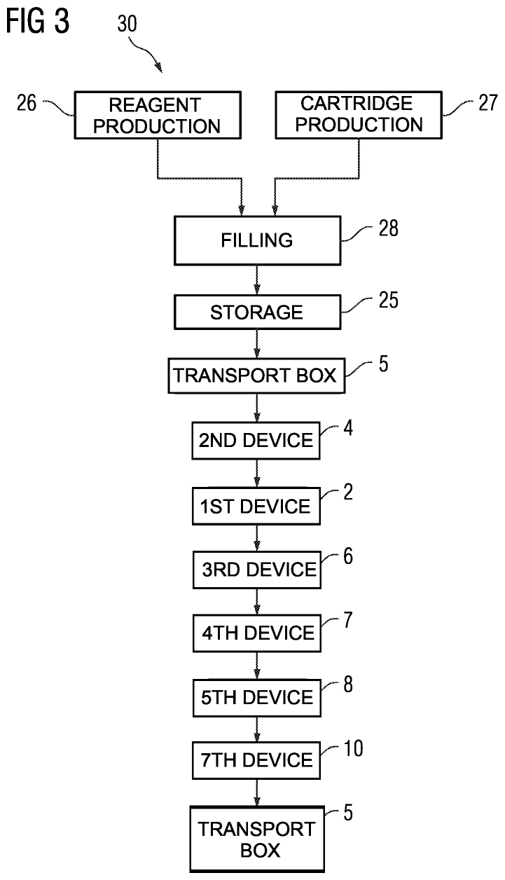

FIG. 3 schematically shows a method (30) for analyzing a sample (23) by means of an automatic analysis system (1) in relation to a factor VIII binding test, which is configured as an enzyme-linked immunosorbent assay (ELISA).

First, preferably test-specific cartridges (3) with compartments for reagents and controls and with cuvettes for the measurements are produced (cartridge production (27)).

Further, the required reagents of an approved test preferably available on the market are produced, preferably unmodified, and added in a ready-to-use form into the compartments of the cartridge (3) (reagent production (26)).

For example, the filling volumes and uses for the individual cuvettes (31 to 52) are as follows.

Cuvette 16 (46) has a filling volume of 8000 μL. Cuvettes 1 to 6 (31 to 36) are intended for carrying out measurements and each have coated cuvette bottoms. Cuvettes 7 to 12 (37 to 42) have a filling volume of 160 μL. Cuvettes 17 to 22 (47 to 52) are empty, i.e., they have a filling volume of 0 μL. Cuvette 13 (43) has a filling volume of 85 μL. Cuvettes 14 and 15 (44 and 45) have a filling volume of 110 μL.

The cuvettes (31 to 52) are each filled with the following substances according to the filling volumes specified above, or their bottom is correspondingly coated.

The cuvette bottoms of cuvettes 1 to 6 (31 to 36) are coated with F(ab′)2—fragments of rabbit antibodies against human VWF (von Willebrand factor).

Cuvettes 7 and 8 (37 and 38) are filled with reagent with recombinant human factor VIII.

Cuvettes 9 and 10 (39 and 40) are filled with reagent with peroxidase-coupled monoclonal mouse antibody against human factor VIII.

Cuvettes 11 and 12 (41 and 42) are filled with reagent with tetramethylbenzidine (TMB).

Cuvette 13 (43) is filled with reagent with H2SO4.

Cuvette 14 (44) comprises control 1 and is filled with human plasma, which contains a known amount of VWF:FVIIIB in the normal range.

Cuvette 15 (45) comprises control 2 and is filled with human plasma, which contains a known amount of VWF:FVIIIB in the pathologically low range.

Cuvette 16 (46) is filled with wash solution, which is used to wash the measurement cuvettes.

Cuvettes 17 to 22 (47 to 52) are empty.

After having carried out the filling (28), the compartments of cuvettes 1 to 22 (31 to 52) of the cartridge (3) are sealed with a film.

The cartridge (3) is frozen and transferred into a central storage unit (25), where it is kept at minus 20 degrees Celsius or lower.

The cartridge (3), or alternatively a plurality of cartridges (3), is subsequently packed into a transport box (5) and transported at minus 20 degrees Celsius or lower to an analysis system (1). An order for a particular selection and number of cartridges (3) respectively initiates the corresponding transfers into the transport box (5) and the transport to the location of the analysis system (1).

At the location of the analysis system (1), the transport box (5) is introduced into the second device (4).

The analysis system (1) takes the cartridge (3) out of the transport box (5) in an automated fashion and brings it into the first device (2) for storage, the temperature of the first device (2) being minus 20 degrees Celsius or lower.

Placing a sample (23) on the analysis system (1) initiates transfer of the cartridge (3) required for this sample (23) into the third device (6).

In the third device (6), the cartridge (3) is thawed by means of the fourth device (7) and preferably regulated to 37 degrees Celsius by means of a water bath.

The cartridge (3) regulated to 37 degrees Celsius is subsequently transferred into the fifth device (8) and the test in question is then performed, including pipetting steps, washing steps and incubation steps. Incubation steps are preferably carried out in the sixth device (9). The various pipetting steps, washing steps and incubation steps take place in the following sequence.
i) 50 μL of sample (23), 50 μL of control 1 and 50 μL of control 2 are respectively pipetted into two of the measurement cuvettes, the covering film being pierced;
ii) incubation for 30 minutes at 37 degrees Celsius;
iii) washing five times with 75 μL of the wash solution in each case;
iv) pipetting 50 μL of reagent a. (reagent with recombinant human factor VIII) into each of the 6 measurement cuvettes;
v) incubation for 30 minutes at 37 degrees Celsius;
vi) washing five times with 75 μL of the wash solution in each case;
vii) pipetting 50 μL of reagent b. (reagent with peroxidase-coupled monoclonal mouse antibody against human factor VIII) into each of the 6 measurement cuvettes;
viii) incubation for 30 minutes at 37 degrees Celsius;
ix) washing five times with 75 μL of the wash solution in each case;
x) pipetting 50 μL of reagent a. (reagent with tetramethylbenzidine (TMB)) into each of the 6 measurement cuvettes;
xi) incubation for 2.5 minutes at 37 degrees Celsius;
xii) pipetting 12.5 μL of reagent e. (reagent with H2SO4 (1M)) into each of the 6 measurement cuvettes;
xiii) incubation for 7.5 minutes at 37 degrees Celsius.

The sample (23) is 0.11 M citrate plasma, which had been diluted with phosphate buffer to a VWF:Ag value of 10% of the standard. The VWF:Ag concentration of the sample needs to have been determined beforehand so that the dilution factor can be established.

The cartridge (3) is subsequently transferred into the seventh device (10) in order to measure the sample preparation and evaluate the measurement results. The extinction of the sample preparation at a wavelength of 450 nm is measured. The evaluation takes place in relation to an electronically transmitted calibration curve which was determined centrally for the respective batch of the cartridge (3) by using precisely the corresponding test procedure.

Further, the used cartridge (3) is transferred into the transport box (5). The transport box is now no longer cooled and is advantageously at room temperature. The cartridge (3) remains in the transport box (5) until the transport box (5) is replaced with a newly delivered, further transport box (5). The newly delivered transport box (5) is in turn loaded with frozen cartridges (3). The used cartridge (3) is preferably disposed of in a way which is as environmentally friendly as possible or delivered for reuse. The transport box (5) which carried the used cartridge (3) is cleaned and prepared for the next transport of further, new cartridges (3).

### LIST OF REFERENCE SIGNS

1 analysis system
2 first device (for storing cartridges at T1)
3 cartridge
T1 first temperature
4 second device (for receiving a transport box)
5 transport box
6 third device (for storing cartridges at T2)
T2 second temperature
7 fourth device (for heating the cartridges from T1 to T2)
8 fifth device (for pipetting at least one liquid)
9 sixth device (for incubating at least one cartridge)
10 seventh device (for measuring at least one sample preparation)
11 test preparation
12 first compartment (of the cartridge with at least one reagent)
13 reagent
14 second compartment (of the cartridge with at least one control)
15 control
16 third compartment (of the cartridge comprising a measurement cuvette)
17 measurement cuvette
T3 third temperature
T4 fourth temperature
18 recess
19 eighth device (for controlling T1, T2, T3, T4)
20 ninth device (for automatically bringing cartridge from 5 to 2)
21 tenth device (for automatically bringing cartridge from 2 to 6)
22 eleventh device (for automatically bringing cartridge from 6 to 5)
23 sample
24 calibration curve
25 central storage unit
26 reagent production
27 cartridge production
28 filling
30 method
31 cuvette 1
32 cuvette 2
33 cuvette 3
34 cuvette 4
35 cuvette 5
36 cuvette 6
37 cuvette 7
38 cuvette 8
39 cuvette 9
40 cuvette 10
41 cuvette 11
42 cuvette 12
43 cuvette 13
44 cuvette 14
45 cuvette 15
46 cuvette 16
47 cuvette 17
48 cuvette 18
49 cuvette 19
50 cuvette 20
51 cuvette 21
52 cuvette 22

1. An automatic analysis system for analyzing a sample, the analysis system comprising:
a first device for storing cartridges at a first temperature, the first temperature less than 268.15 kelvin,
a second device for receiving a transport box,
a third device for storing cartridges at a second temperature, the second temperature being more than 273.15 kelvin,
a fourth device for heating the cartridges from the first temperature to the second temperature,
a fifth device for pipetting at least one liquid,
a sixth device for incubating at least one cartridge, and
a seventh device for measuring at least one sample preparation of the sample, wherein the cartridges each comprise at least one first compartment with at least one reagent, at least one second compartment with at least one control, and at least one third compartment comprising a measurement cuvette,
wherein a measurement of the test preparation can be made with the seventh device in the measurement cuvette,
wherein at least one cartridge can be delivered to the analysis system,
wherein the cartridge has a third temperature, the third temperature less than 268.15 kelvin, and
wherein the transport box is configured to transport at least one cartridge at the third temperature.

2. The automatic analysis system as claimed in claim 1, wherein the transport box is further configured to transport at least one cartridge at a fourth temperature, the fourth temperature ranging from 233.15 to 323.15 kelvin.

3. The automatic analysis system as claimed in claim 1, wherein the third device at least partially comprises the fourth device, the fifth device, the sixth device or the seventh device.

4. The automatic analysis system as claimed in claim 1, wherein the second temperature is a predetermined, constant temperature between 293.15 and 315.15 kelvin.

5. The automatic analysis system as claimed in claim 1, wherein the first temperature ranges from 255.15 to 245.15 kelvin.

6. The automatic analysis system as claimed in claim 1, wherein the liquid comprises a sample liquid.

7. The automatic analysis system as claimed in claim 1, wherein the fifth device comprises a pipetting needle for pipetting samples, reagents or a dilution medium, the dilution medium comprising a buffer solution.

8. The automatic analysis system as claimed in claim 1, wherein the second device comprises a recess, the recess configured to receive at least one transport box.

9. The automatic analysis system as claimed in claim 2, wherein the analysis system comprises an eighth device for controlling the first, second, third or fourth temperature.

10. The automatic analysis system as claimed in claim 1, wherein the analysis system comprises a ninth device for automatically bringing at least one cartridge out of a transport box contained in the second device and into the first device.

11. The automatic analysis system as claimed in claim 1, wherein the analysis system comprises a tenth device for automatically bringing at least one cartridge out of the first device and into the third device.

12. The automatic analysis system as claimed in claim 1, wherein the analysis system comprises an eleventh device for automatically bringing at least one cartridge out of the third device into a transport box contained in the second device.

13. A transport box for an automatic analysis system as claimed in claim 1 for transporting at least one cartridge to or from the analysis system, wherein the cartridge comprises at least one first compartment with at least one frozen reagent, at least one second compartment with at least one control, and at least one third compartment, comprising a measurement cuvette, the transport box containing at least one cartridge.

14. A cartridge which can be transported via a transport box to or from an automatic analysis system as claimed in claim 1, wherein the cartridge comprises at least one first compartment with at least one frozen reagent, at least one second compartment with at least one control, and at least one third compartment comprising a measurement cuvette.

15. (canceled)

16. A method for analyzing a sample in an automatic analysis system by using a cartridge and a transport box, the method comprising the following steps:
delivering the cartridge in the transport box to the automatic analysis system, the cartridge having a first temperature less than 268.15 kelvin,
bringing the transport box with the cartridge into a second device,
removing the cartridge from the transport box and delivering it into a first device with a ninth device, or delivering the cartridge into the first device,
removing the cartridge from the first device and delivering it into a third device with a tenth device, the third device for storing cartridges at a second temperature, the second temperature higher than 273.15 kelvin, the tenth device for automatically bringing at least one cartridge out of the first device and into the third device,
heating the cartridge from the first temperature to the second temperature using a fourth device, the fourth device for heating the cartridge from the first temperature to the second temperature,
pipetting at least one sample, at least one reagent and at least one control using a fifth device to form a test preparation,
incubating the test preparation in the cartridge using a sixth device, for incubating at least one cartridge,
measuring the test preparation using a seventh device in the measurement cuvette of a third compartment of the cartridge, and
evaluating the measurement of the test preparation by using at least one calibration curve, which has been transmitted electronically to the analysis system.

17. The method for analyzing a sample as claimed in claim 16, wherein the method comprises carrying out a factor VIII binding test.

18. The method for analyzing a sample as claimed in claim 16, further comprising:
bringing the cartridge out of the third device into the transport box using an eleventh device, and
transporting the cartridge in the transport box to a disposal station, the cartridge having a fourth temperature ranging from 233.15 to 323.15 kelvin.

19. The automatic analysis system as claimed in claim 1, wherein the first temperature is less than 263.15 kelvin.

20. The automatic analysis system as claimed in claim 1, wherein the second temperature is between 308.15 and 312.15 kelvin.

21. The automatic analysis system as claimed in claim 2, wherein the fourth temperature ranges from 288.15 to 298.15 kelvin.
